(19) United States
(12) Patent Application Publication
(10) Pub. No.: US 2020/0138879 A1
(43) Pub. Date: May 7, 2020

(54) TREATMENT OF INFANT COLIC

(71) Applicant: SOCIETE DES PRODUITS NESTLE S.A., Vevey (CH)

(72) Inventors: Clara Lucia Garcia-Rodenas, Forel (CH); Gabriela Bergonzelli Degonda, Bussigny (CH)

(21) Appl. No.: 16/610,274

(22) PCT Filed: May 2, 2018
(86) PCT No.: PCT/EP2018/061101

§ 371 Date: Nov. 1, 2019

(30) Foreign Application Priority Data
May 2, 2017 (EP) 17169822.8

(51) Int. Cl. A61K 35/745; A61K 9/00; A61P 1/14

(52) U.S. Cl. CPC A61K 35/745; A61P 1/14; A61K 9/0095

(57) ABSTRACT

Methods and uses of B. longum strain ATCC-999 for the treatment or prophylaxis of infant colic are disclosed.

### FIELD OF INVENTION

The present invention relates to a probiotic and a composition thereof use in the treatment or prophylaxis of infant colic. In particular the present invention relates to the treatment or prophylaxis of infant colic.

### BACKGROUND

Infant colic is characterized by prolonged episodes of crying (especially a high pitched scream) and typically affects about 10-30% of babies. Infant colic can affect breast-fed and non-breast-fed infants. A diagnosis of colic is made after ruling out other possible causes.

These episodes may also include irritability, drawing up of the legs, and grimacing and tend to be worse in the late afternoon or evening. Typically, infant colic episodes are non-responsive to the usual comforting measures, such as cuddling or feeding. Infant colic often appears 3-4 weeks after birth and typically reaches a peak around 6 weeks, and in most case, resolves itself around 12-16 weeks after birth, or in the case of babies born prematurely 12-16 weeks after term. Colic only rarely affects infants at 6 months or beyond. In preterm infants, these periods are calculated from the full-term equivalent. According to the definition of Wessel [Wessel, M. A., J. C. Cobb, et al. (1954), “Paroxysmal fussing in infancy, sometimes called colic.” Pediatrics 14(5): 421-435], colic relates to paroxysms of crying for 3 or more hours per day for 3 days or more per week during a period of at least 3 weeks for three or more hours per day, on three or more days per week for three or more consecutive weeks. By comparison, on average, an infant without colic cries on average of around 2 hours a day, with the duration reaching a maximum at around 6 weeks.

A particularity of the cries of the first weeks of life is that they concentrate in late afternoon. The peak shape of the crying behavior found in colicky and non-colicky babies, its circadian rhythm and the fact that colicky babies cry longer than non-colicky babies makes it highly possible that the prolonged episodes of crying reflect a difference in individual central nervous system [Barr, R. G.—“Changing Our Understanding of Infant Colic”—Arch. Pediatr. Adolesc. Med. 2002, 156(12), 1172-1174)]. Since bouts of cries concentrate in late afternoon when colicky babies seem to be over alert instead of showing signs of tiredness (Savino, F, “Focus on infantile colic”—Acta Paediatr. 2007, 96(9):1259-1264), it has been hypothesized that there is a relationship between infantile colic and the development of the circadian rhythm.

Possible causative facts in colic include gastrointestinal disorders (e.g. improper feeding, lack of burping, sensitivity to cow's milk protein in infant formula-fed babies, lactose intolerance or malabsorption) and neurological development disorders manifesting in e.g. abdominal cramps and hyperperistalsis. However, the aetiology of infant colic is not well understood.

A number of treatments have been proposed or used for treating colic. These include pharmacological and non-pharmacological interventions, such as dietary changes such as use of hypoallergenic or lactose-free milk, behavioural training, massage and physical movement. Pharmacological interventions have included the administration of anticholinergic drugs for example to relieve cramps and other abdominal disorders, and sedatives. However, both anticholinergic drugs and sedative drugs potentially have serious side effects, which severely limit their use for treating colic. Thus, presently there remains no cure for colic. In view of this, and since infant colic is not normally regarded to be a serious or life-threatening medical condition, the most common treatment for infant colic at present is to manage the colic symptoms without any form of therapy and essentially allow the infant to grow out of the condition.

However, the frequency and duration of the crying associated with colic can be extremely distressing for the parent or caregiver, particularly as it occurs in the early stages of the infant's life. Infant colic can severely impact on the quality of life of the parent or caregiver, leading to anxiety, fatigue, a disruption to family life, and may be detrimental to their ability to bond with the infant. Colic is also a leading cause of the baby shaken syndrome and is associated with increased risk of functional gastrointestinal disorders, migraine and behavioural alterations in childhood. Moreover, infant colic may result in multiple visits to the doctor in order to rule out other underlying health issues, and contributes significantly to healthcare costs, particularly as a result of unnecessary dietary changes, medical prescriptions for acid reflux or for special infant formulae, and early termination of breastfeeding.

Therefore, there is a great need to provide compositions and methods for treating or for preventing infant colic, which are safe and easy to administer. An object of the present invention is to address this need and to provide treatment of infant colic and compositions for treating infant colic.

DESCRIPTION OF FIGURES

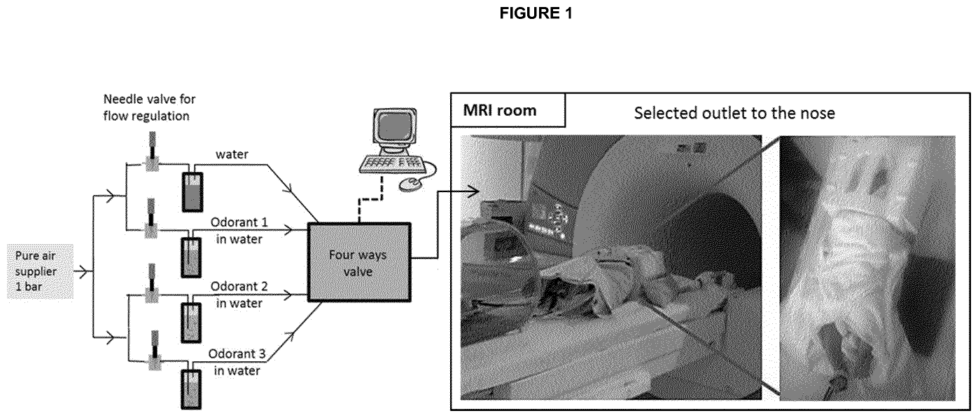

FIG. 1: Apparatus set-up for olfactory stimulation experiments showing a four-way delivery system for the 3 odours (cabbage, banana and eucalyptol) and a neutral odour (water) used in Example 1, which is carried out during magnetic resonance imaging (MRI).

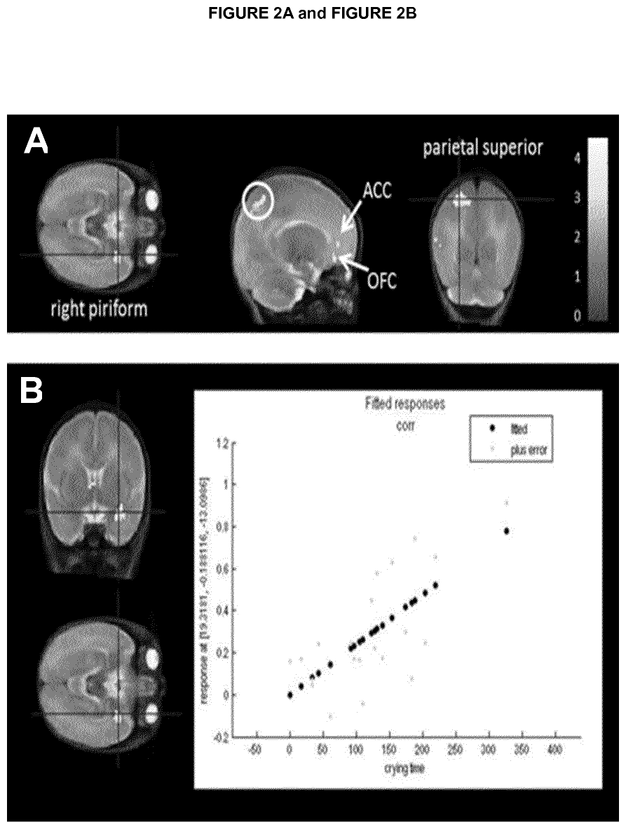

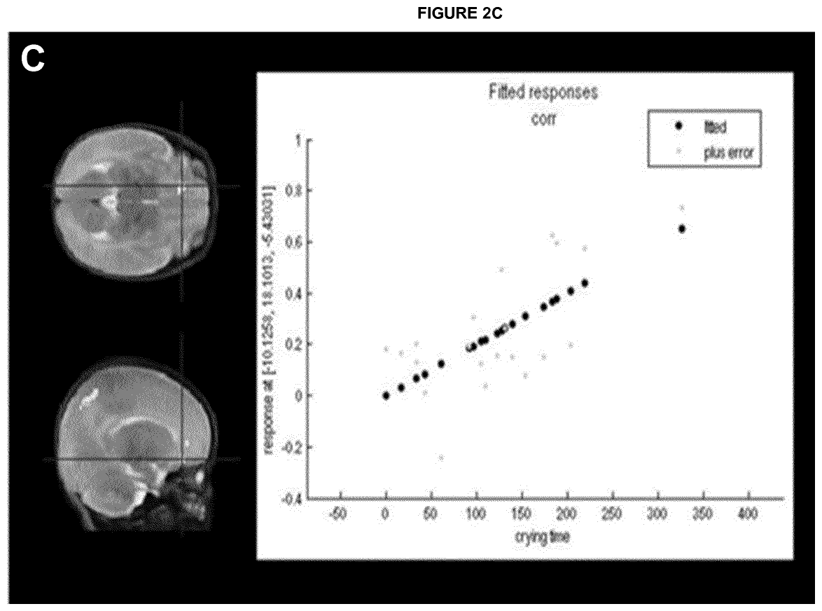

FIGS. 2A-2C: The plot shows a linear regression for the olfactory stimulation using cabbage odour (n=21) showing significant positive correlation (p<0.005, uncorrected) between the mean daily crying time and the activation of:
(A): the right piriform cortex, the left orbitofrontal cortex (OFC), the anterior cingulate cortex (ACC), the left superior parietal lobule and the precuneus (t=3+). Activations are overlaid on the T2-weighted newborn's template. Color/shaded bar (top right of the upper figure) indicates t-values scale (where dark=0 and white=5+); and
(B): Plot of the linear regression at the coordinate position (on the maximum value) in the right piriform cortex (x-axis=crying time in minutes; y-axis=response at coordinates 19.3181, −0.188116, −13.0986). The large plot markers are fitted values and the feint plot marks include the actual values; and
(C): in the left OFC (x-axis=crying time in minutes; y-axis=response at coordinates −10.1258, 18.1013, −5.43031). The large plot markers are fitted values and the feint plot marks include the actual values.

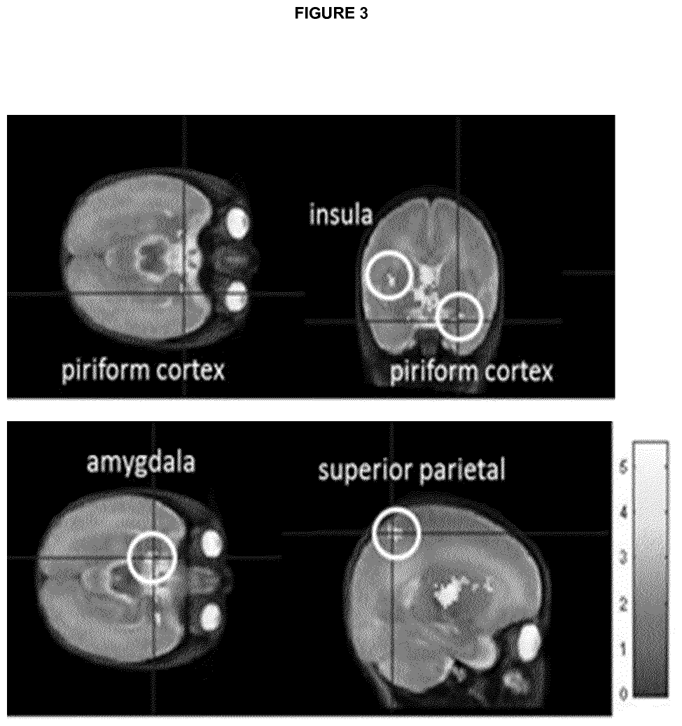

FIG. 3: Group comparison for the cabbage condition: When exposed to cabbage odour, colicky infants (n=11) exhibited statistically greater activation in the left amygdala and left insula, as well as in other cortical regions such as the left superior parietal lobule, than did non-colicky infants (p<0.005, uncorrected; two-sample t-test). Activations are overlaid on the T2-weighted newborn's template (t=4+). Color/shaded bar indicates t-values scale (where dark=0 and white=5+).

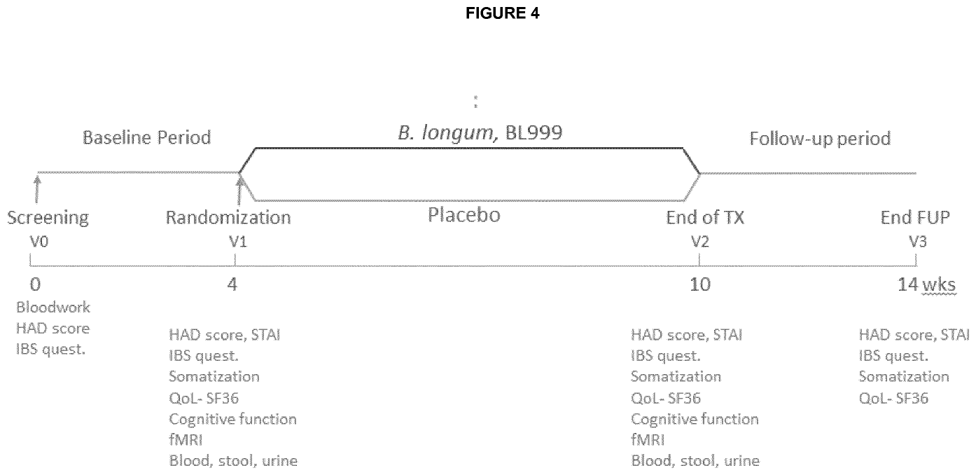

FIG. 4: Design of the clinical trial of Example 2 (administration of B. longum ATCC BAA-999 to IBS patients)

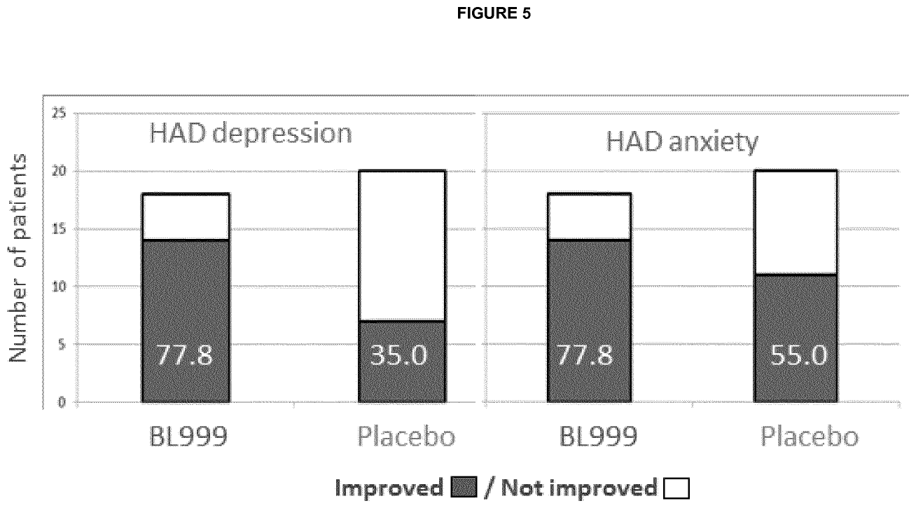

FIG. 5: Graphs demonstrating the primary outcome from administration of B. longum ATCC BAA-999, improvement in HAD (hospital anxiety and depression) depression and HAD anxiety dichomotous scores.

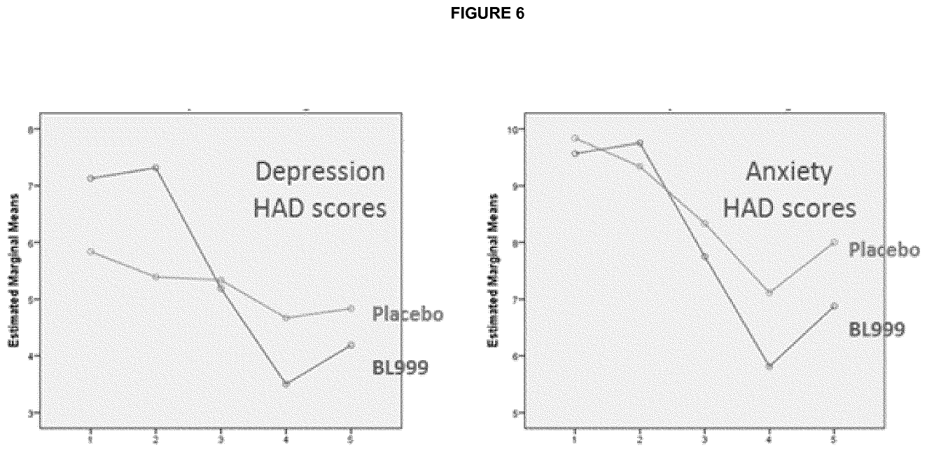

FIG. 6: Graphs demonstrating the secondary outcome from administration of B. longum ATCC BAA-999, improvement in depression and anxiety continuous scores.

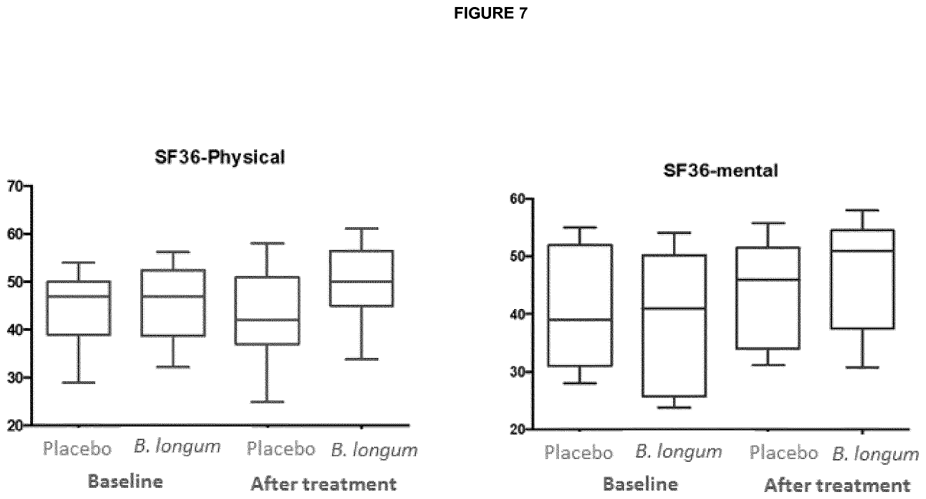

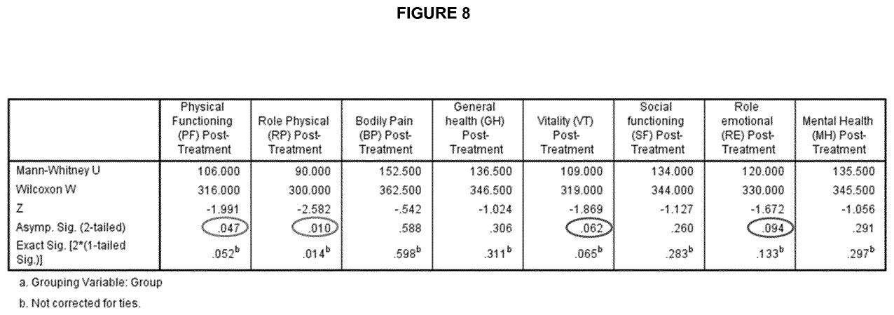

FIGS. 7 AND 8: Graphs (FIG. 7) and table (FIG. 8) demonstrating that administration of B. longum ATCC BAA-999 significantly improved the physical global domain as well as general physical health (physical functioning) and problems with work of other daily activities (role physical) and resulted in an improvement trend in the mental subdomains of vitality and role emotional.

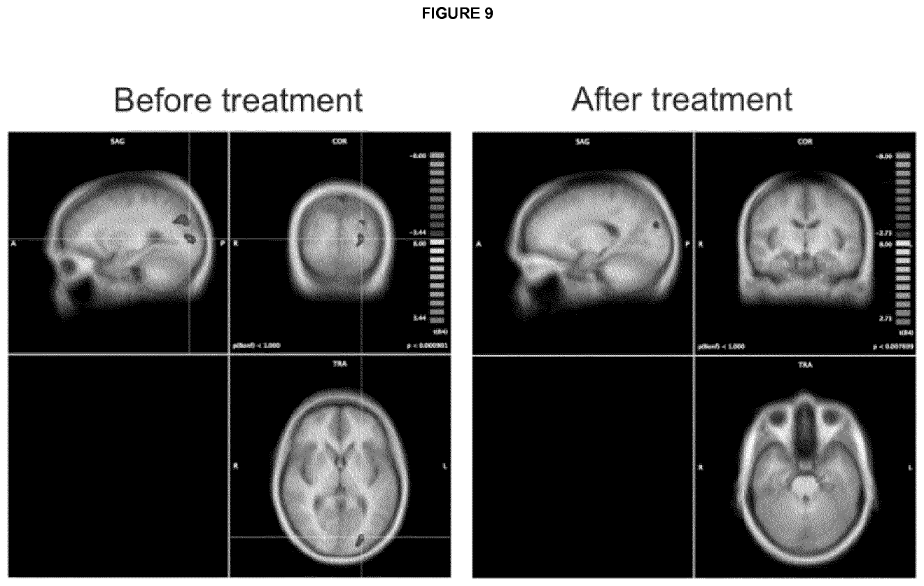

FIG. 9: fMRI images demonstrating greater engagement of the visual association and parietal cortices in the group administered B. longum ATCC BAA-999 relative to the placebo group and lesser engagement of brain centers involved in emotion and mood (amygdala and fronto-limbic region) in the group administered B. longum ATCC BAA-999 relative to the placebo group.

### SUMMARY OF THE INVENTION

We have investigated a hypothesis that the excessive crying in otherwise healthy infants which characterises infant colic, may represent the upper end of the spectrum of early developmental crying behaviour seen in all infants, and that this may be due to differences in central nervous system functioning with heightened sensory reactivity.

In our investigations, babies with excessive crying time present more pronounced cerebral activity in various brain regions including the amygdala, orbitofrontal cortex and anterior cingulate cortex, shortly after birth. The cerebral activity is particularly seen at 5-6 weeks, i.e. when infant colic typically reaches its peak level. The amygdala is a brain area involved in emotion regulation, as well as in pain response and visceral/body sensation. This early difference in activation after sensory stimulation could explain the difference in behaviour between colicky and non-colicky babies.

We have also investigated the effect of administration of Bifidobacterium longum (BL999) on the activity in certain regions of the brain. Our investigations show that heightened activity, particularly in the amygdala (i.e., the same region of heightened activity seen in the colicky infants), in a model for anxiety in irritable bowel syndrome (IBS) patients is attenuated following oral administration of BL999.

The present invention therefore provides the use of a particular probiotic, namely Bifidobacterium longum ATCC BAA-999 in the treatment or prophylaxis of infant colic.

In accordance with another aspect of the present invention, Bifidobacterium longum ATCC BAA-999 may be used in reducing cerebral activity in an infant with colic.

In another aspect of the present invention, there is provided a use of Bifidobacterium longum ATCC BAA-999 or a composition thereof for reducing the frequency and/or duration of crying episodes in an infant with colic.

A further aspect of the present invention provides a method of reducing the frequency and/or duration of crying episodes in a subject with infant colic, wherein the method comprises administering Bifidobacterium longum ATCC BAA-999 or a composition comprising Bifidobacterium longum ATCC BAA-999 to the subject.

The invention further provides a composition comprising Bifidobacterium longum ATCC BAA-999 for use in the treatment or prophylaxis of infant colic.

The invention further provides a composition comprising Bifidobacterium longum ATCC BAA-999 for use in reducing cerebral activity in an infant with colic.

Also provided is a composition comprising Bifidobacterium longum ATCC BAA-999 for use in reducing the frequency and/or duration of crying episodes in an infant with colic.

The compositions of the present invention may be used in the treatment or prophylaxis of infant colic, or in reducing cerebral activity in an infant with colic; or in reducing the frequency and/or duration of crying episodes in an infant with colic.

### DETAILED DESCRIPTION OF THE INVENTION

As used herein, unless otherwise indicated, the term “Bifidobacterium longum ATCC BAA-999” is used interchangeably with “B. longum ATCC BAA-999” and “BL999”, and includes the bacterium, parts of the bacterium, a cell growth medium with the bacterium or parts of the bacterium or a cell growth medium in which Bifidobacterium longum ATCC BAA-999 was cultivated. Preferably, Bifidobacterium longum ATCC BAA-999 refers to the bacterium, parts of the bacterium, or a cell growth medium containing the bacterium or parts of the bacterium. Bifidobacterium longum ATCC BAA-999 may be present as viable bacteria, as non-replicating bacteria, or as a mixture thereof. Bifidobacterium longum ATCC BAA-999 (BL999) may be obtained commercially from specialist suppliers, for example from Morinaga Milk Industry Co. Ltd. of Japan under the trade mark BB536. Bifidobacterium longum ATCC BAA-999 (BL999) may be cultured according to any suitable method. It may be used in accordance with the invention, for example, in a freeze-dried or in a spray-dried form.

The term “probiotic” means microbial cell preparations or components of microbial cells with a beneficial effect on the health or well-being of the host (Salminen S, Ouwehand A. Benno Y. et al “Probiotics: how should they be defined” Trends Food Sci. Technol. 1999:10 107-10).

As used herein, the term “prebiotic” means food substances intended to promote the growth of probiotic bacteria in the intestines.

As used herein, the term “infant” means a person not more than 12 months of age.

As used herein, a reference to a period of x weeks “from term-adjusted birth” or “full-term equivalent” is used in relation to preterm infants. In this context, the correction from preterm age to the full term equivalent is based on 40 weeks as the full-term age. Thus in a preterm infant as defined above, a period of x weeks from term-adjusted birth means that the time period of x weeks is calculated starting from the infant's 40th week. Thus, for a preterm infant born at 36 weeks (i.e. 40−36=4 weeks preterm), a period of 6 weeks from “term-adjusted birth” means an adjusted period of 6+4=10 weeks from birth.

As used herein, unless otherwise indicated, in the context of the present invention, the term colic is defined in accordance with the “Rome III” criteria (Hyman, P. E.—“Childhood Functional Gastrointestinal Disorders: Neonate/Toddler”—Gastroenterology, 2006; 130:1519-1526), which requires all of the following in infants from birth to 4 months of age:
1. Paroxysms of irritability, fussing, or crying that start and stop without obvious cause
2. Episodes lasting 3 or more hours per day and occurring at least 3 days per week for at least 1 week
3. No failure to thrive.

The term “infant formula” refers to a foodstuff intended for particular nutritional use by infants during the first year of life and satisfying by itself the nutritional requirements of this category of person, as defined in European Commission Directive 2006/141/EC of 22 Dec. 2006. In the context of the present invention, the term “infant formula” includes a liquid ready-to-use infant formula, or an infant formula in a concentrate or powdered form for dilution/reconstitution into a liquid infant formula (preferably using water) to form a liquid composition suitable for feeding an infant. Preferably, the infant formula of any aspect or embodiment of the present invention is in the form of a powder. The infant formula may be nutritionally complete, or can be a complementary infant formula which can be used in conjunction with human milk.

The term “maternal lactation supplement” refers to a composition which is for administration to the mother during at least part of the period in which the infant is ingesting breastmilk from the mother. The infant may be fully breastfed, or may be fed on a combination of breastmilk and infant formula.

The term “infant supplement” refers to a composition which is suitable for administration to the infant. The infant supplement is typically used as a complement to breastmilk and/or an infant formula. The infant supplement may be in liquid form, or may be in the form of a powder or concentrate, which can be diluted or reconstituted to form a liquid for ease of administration to the infant.

As used herein, the term “treatment” means management of the infant colic, for example to cure, ameliorate or to stabilize the infant colic.

As used herein, the term “prophylaxis” means preventing or inhibiting infant colic.

In the course of our olfactory stimulation experiments, we have identified areas of increased cerebral activity (e.g. in the amygdala and other areas involved with emotional and anxiety behaviour) in colicky infants when exposed to particular odours. These areas were significantly more active in the infants with colic compared with infants without colic. Our experiments on patients with irritable bowel syndrome have shown that Bifidobacterium longum ATCC BAA-999 can be used to reduce cerebral activity in the amygdala, i.e. the same area having heightened activity in infants with colic.

Thus, in a first aspect, the present invention provides Bifidobacterium longum ATCC BAA-999 for use in the treatment or prophylaxis of infant colic.

In any aspect or embodiment of the present invention, the term “infant colic” is preferably defined in accordance with the Rome III criteria as described above.

According to a further aspect of the present invention, there is provided Bifidobacterium longum ATCC BAA-999 for use in reducing cerebral activity, for example reducing over activity or normalising activity, in an infant with colic. Particularly the reduction in cerebral activity is in the amygdala. The reduction in cerebral activity can be determined by a decrease in the frequency and/or duration of colic episodes in the infant, or by magnetic resonance imaging comparisons.

Over activity may mean that activity is increased in comparison to an infant that does not suffer with colic.

The invention further provides the use of Bifidobacterium longum ATCC BAA-999 or a composition thereof, for reducing frequency and/or duration of episodes of irritability, fussing and/or crying in a subject with infant colic. The invention further provides a method of reducing frequency and/or duration of episodes of irritability, fussing and/or crying in a subject with infant colic, comprising administering Bifidobacterium longum ATCC BAA-999 or a composition thereof to the subject.

In any aspect or embodiment of the present invention, the use or methods disclosed herein can be in an infant having colic and has been identified as having an increased cerebral activity, preferably wherein the infant has been identified as having an increased cerebral activity in the amygdala. The increased cerebral activity can be determined by the MRI methods disclosed herein, or by any other suitable method.

Bifidobacterium longum ATCC BAA-999 can be used to treat an infant having colic when there has been no formal diagnosis of an underlying disease.

In any aspect or embodiment of the present invention, the Bifidobacterium longum ATCC BAA-999, or a composition comprising the same, is preferably administered to the infant in an amount of: about 1×10⁸ CFU or more per day, about 5×10⁸ CFU or more per day, about 1×10⁹ CFU or more per day, about 5×10⁹ CFU or more per day, about 1×10¹⁰ CFU or more per day, about 5×10¹⁰ CFU or more per day, about 1×10¹¹ CFU or more per day, or about 5×10¹¹ CFU or more per day, about 1×10¹² CFU or more per day, or about 5×10¹² CFU or more per day.

Alternatively, in any aspect or embodiment of the present invention, the Bifidobacterium longum ATCC BAA-999, or a composition comprising the same, is preferably administered to the infant in an amount of: about 1×10⁸ to about 5×10¹¹ CFU per day, about 5×10⁸to about 5×10¹¹ CFU per day, about 5×10⁸to about 5×10¹¹ CFU per day, about 1×10⁹ to about 5×10¹¹ CFU per day, about 5×10⁹ to about 5×10¹¹ CFU per day, about 1×10¹⁰ to about 5×10¹¹ CFU per day, or about 1×10¹⁰ to about 1×10¹¹ CFU per day, about 1×10 CFU or more per day, or about 5×10¹² CFU or more per day. Most preferably, the daily dose of Bifidobacterium longum ATCC BAA-999 is about 1×10¹⁰ to about 1×10¹¹ CFU per day.

Compositions of the invention which are in the form of an infant formula may be prepared containing the Bifidobacterium longum ATCC BAA-999 at a concentration in order to provide these daily amounts to the infant.

Compositions of the present invention in the form of an infant supplement, or breast milk supplement, may be prepared so as to provide Bifidobacterium longum ATCC BAA-999 in an amount that is equivalent above daily dosage ranges to the infant, either as a single daily supplement or as a supplement to be administered more than once per day (twice daily, three times daily, or four times daily). Infant supplements or breastmilk supplements are preferably prepared in the form of a liquid or powder (e.g. a freeze dried or spray dried powder). The powder can be dispersed/dissolved into a small volume of liquid (for example, water, breast milk, an infant formula) which can be easily administered orally to the infant (e.g. via a syringe, a spoon, etc.).

In accordance with any aspect or embodiment of the present invention, the Bifidobacterium longum ATCC BAA-999, or a composition comprising the same, may be administered as soon after birth as possible, for example from: 0, 1, 2, 3, 4, 5, 6 or 7 days from birth, more preferably within a period of: 0 to 5 days, 0 to 4 days, 0 to 3 days or 0 to 2 days, from birth. Although not necessary, in the case of a preterm infant, these periods can be adjusted to the full-term equivalent. Thus, for both term and preterm infants, the Bifidobacterium longum ATCC BAA-999, or a composition comprising the same, is preferably administered as soon as possible after birth. For example if the infant is formula fed, an infant formula comprising Bifidobacterium longum ATCC BAA-999 in accordance with the present invention may be used as the starter formula and the sole nutritional source. Alternatively an infant formula comprising Bifidobacterium longum ATCC BAA-999 in accordance with the present invention may be used as a complementary nutritional source for a breast-fed infant.

Alternatively, the administration can be initiated as soon as signs of colic are observed, or may be initiated following a diagnosis of colic from a medical professional, or when the infant has been determined to show pronounced cerebral activity, for example, in the amygdala by the methods disclosed herein.

The Bifidobacterium longum ATCC BAA-999 can be administered according to any aspect or embodiment of the present invention over the duration of the period which infants have colic—typically over a period of: 0-18 weeks, 0-16 weeks, 0-12 weeks, 1-12 weeks, or 1-8 weeks, from birth. In the case of a preterm infant, these periods are preferably adjusted to full-term equivalent as described above.

In any aspect or embodiment of the present invention the Bifidobacterium longum ATCC BAA-999 can comprise administration of Bifidobacterium longum ATCC BAA-999 or a composition thereof to the lactating mother, preferably from: 0, 1, 2, 3, 4, 5, 6 or 7 days from birth of the infant. By lactating mother, it is meant that the mother is providing breast milk for ingestion by the infant either directly or indirectly. The composition may be administered to the lactating mother from 0, 1, 2, 3, 4, 5, 6 or 7 days from birth of the infant, until: about 2 to about 24 weeks, about 4 to about 20 weeks, about 4 to about 20 weeks, about 8 to about 16 weeks, or about 12 to about 16 weeks from birth, or from term-adjusted birth. Compositions for maternal administration can be in the form of a solid dosage form, such as a tablet, capsule, sachet, powder or can be in the form of a liquid or paste. Alternatively, compositions for maternal administration can be in the form of a food product (e.g. a dehydrated soup, a meal replacement, a nutritional bar, a meal replacement).

Compositions comprising Bifidobacterium longum ATCC BAA-999 can be used for:
the treatment or prophylaxis of infant colic;
reducing cerebral activity, preferably in the amygdala, in an infant with colic;
or in reducing the frequency and/or duration of episodes of irritability, fussing and/or crying in a subject with infant colic.

Preferably, compositions according to any aspect or embodiment of the present invention do not comprise a fructooligosaccharide (FOS). Alternatively, compositions according to any aspect or embodiment of the present invention do not comprise a galactooligosaccharide (GOS) in a concentration of more than 8 g/L, or more than 7 g/L, or more than 6 g/L, or more than 5 g/L, or more than 4 g/L, or more than 3 g/L, or more than 2 g/L, or more than 1 g/L, or more than 500 mg/L, or more than 150 mg/L (said concentration may be the concentration upon reconstitution of a composition e.g. with milk or water). Said compositions optionally also do not contain FOS. As a further alternative, compositions according to any aspect or embodiment of the present invention do not comprise a prebiotic and/or a fermentable or a non-digestible oligosaccharide. More preferably compositions according to any aspect or embodiment of the invention do not comprise FOS, and/or GOS in a concentration of more than 8 g/L. These oligosaccharides (GOS and short chain FOS) are generally either indigestible in the gut due to the absence of appropriate enzymes, and are fermented by the gut microbiota and produce gas. Such oligosaccharides may exacerbate the colic symptoms.

The present invention encompasses the use a composition according to any aspect or embodiment as described herein: in the treatment or prophylaxis of infant colic; in reducing cerebral activity, preferably in the amygdala, in an infant with colic; in reducing frequency and/or duration of episodes of irritability, fussing and/or crying in a subject with infant colic.

The invention will now be further described by way of the following, non-limiting examples.

### EXAMPLES

### Example 1

### Olfactory experiments

### Materials and Methods

### Subjects and Stimulation

36 full-term infants (Gestational age: 39.7 weeks, SD=0.97) without signs of neurological disorders participated to the MRI experiment of this study. Neonates were tested between their 1st and 7th day after birth, during natural sleep or while resting quietly in the scanner, without any sedation. Exposure to olfactory stimuli was used as discussed below.

### Stimulation Protocol

Three odorant stimuli were selected for the experiment: (1) rotten cabbage like (dimethyl trisulphide, DT), (2) banana like (isoamyl acetate, IAA) and (3) eucalyptol (EU), a bimodal odor. All odorants have the food grade label and were prepared by Nestlé Flavors Corporation.

Each odorant was diluted (DT:0.5 mg/l, IAA: 25 mg/l, EU: 50 mg/l) into a 500 mL sterile water humidification bottle (Covidien™) and delivered using a home-made four-way odorant delivery system (three odorants and neutral) with a constant humidity rate at a continuous air flow of 0.4 L/min. Concentrations for each odor were adapted to obtain the adequately perceived odor at the outlet. A multiposition actuator (Valco

Instruments Co. Inc.) was controlled using the serial port of the stimulation computer to deliver each odorant independently during 20 seconds in a pseudo-randomized order to minimize interaction effects between substances. Each odorant stimulations was repeated 5 times per run and was separated with 20 seconds of odorless stimulation from a water flask with sterile water (neutral condition). This block-design stimulation allowed us to be less sensitive to the breathing, which introduces movement artifacts.

The three odors and the neutral stimulation were transported from the control room to the MRI through a 5 meters polyetheretherketone (PEEK) tube (inner diameter 1 mm). The extremity of the PEEK tube is attached to the MRI coil such as the air flow directly arrived to both nostrils simultaneously. The propagation delay of the odorant into the tube was of 7 seconds. This delay was checked for each odorant at the beginning of each experiment to detect a potential leakage or malfunction of the system. The temperature in the MRI room was regulated at 20° C. whereas the temperature in the control room was kept at 18° C. to avoid condensation into the tube.

Alternating exposure to neutral odor (water) and to test odors administered in a pseudo-randomised order, was done during MRI using a block paradigm of 20 sec, and a home-made four-way delivery system with a delivery flow rate of 0.4 l/min (FIG. 1).

Of the 36 infants enrolled in the study, 15 were excluded from the analysis due to technical difficulties or excessive motion during the MRI, health issues unrelated to the study, or incomplete crying diaries. A total of 21 infants completed the study.

### MRI Acquisition

All infants were fed immediately before testing to increase the likelihood that they would sleep or stay quiet through the entire procedure.

When infants were fed and quiet, they were swaddled in a blanket, protections were placed on the ears and they were set up in a vacuum pillow longer than baby's body that surrounded the head to prevent from movements.

When infant correctly placed in the scanner with headcoil, the outlet of the PEEK tube for odors delivery was fixed on the border of the headcoil, according to the position of the infant's head, in front of the two nostrils.

Infant's behaviour during experiment was monitored using pulse oxymetry, a camera, a microphone and by the presence of a nurse during the entire MRI acquisition. Scanning was interrupted immediately if the infants became restless.

The acquisition was performed in a 3T MRI (Siemens Magnetom Trio, Erlangen, Germany) using an 8-channel neohead coil (LMT medical systems, Lubeck, Germany).

Before each MRI acquisition, the experimenter controlled the timing of delivery for each odorants and the two pneumatic valves.

fMRI acquisition and pre-processing:
350 functional image
8-channel head coil
3T MRI (Siemens Trio, Erlangen, Germany
Echo-Planar Imaging (repetition time=1800 ms, echo time=25 ms, 30 slices, voxel size=2.2×2.2×3.5 mm³
A T2-weighted structural image acquired for anatomical reference (113 coronal slices, voxel size=0.78×0.78×1.2 mm³)
Head motion: Sessions without motion>6 mm and including a min. of 2 repetitions of each condition were used for analysis
   fMRI Preprocessing

The pre-processing of the functional images was performed with SPM8 (Wellcome Department of Imaging Neuroscience, UCL, London, UK) and included: (i) realignment, (ii) slice timing, (iii) rigid-body co-registration of functional images on the T2 structural image, (iv) normalization of subject anatomical T2 image (1×1×1mm) and EPI (2×2×2mm) to a T2 template of newborns created from 28 infant's anatomical T2 image acquired in this study with an isotropic Gaussian kernel full width at the half maximum, (v) spatial smoothing (6 mm).

To accommodate the high level of motion in infants, images with frame wise displacement superior to 6 mm for the olfactory experiment, as well as one previous and two following images were excluded Power, Barnes et al. Steps toward optimizing motion artifact removal in functional connectivity MRI; a reply to Carp Neuroimage. 2013 1;76:439-41.

1st Level Analysis

Functional time-series were analyzed voxel by voxel with a general linear model (GLM). The six realignment parameters and their Volterra expansion were reduced using singular value decomposition (SVD). The NC first SVD components explaining at least 99% of the variance, or the first six SVD components if NC>6, were included into the GLM model as covariate regressors. This SVD reduction allows the consideration of the 24 realignment parameters to remove any residual motion-related variance without decreasing too much the number of degrees of freedom while ensuring the orthogonality of the model. Block stimulation design for each odor was convolved by the canonical hemodynamic response function (HRF) and used as regressor.

Sessions without motion, including a minimum of 2 repetitions of each condition were used for the analysis and the regressors associated to odorant stimulation and to confound were cut accordingly. Four infants were rejected due to excessive motions and all subsequent analyses were done in 24 infants.

### 2nd Level Analysis

A second level analysis was performed on the group (Random-effect analysis) of 24 infants using a one-sample t-test, with a threshold of significance set at minimum at: p<0.005 with a minimum of 10 voxels extent, to identify regions involved in the processing of each odorants separately contrasted with the neutral condition (water).

### Crying Behaviour at 6 Weeks:

The parents kept a crying diary for 14 days starting from week 5-6 of life [adapted from Barr, R. G., M. S. Kramer, et al. (1988). “Parental diary of infant cry and fuss behaviour.” Arch Dis Child Fetal Neonatal Ed 63(4): 380-387]. Each 24 hr period was divided into 96 intervals of 15 minutes during which parents noted whether their baby was awake and calm, crying, fussing or sleeping.

A baby was considered as colicky, if the baby cried more than 3 hours a day for at least 3 days within a 7 day period according to Rome III criteria [Hyman, P. E.—“Childhood Functional Gastrointestinal Disorders: Neonate/Toddler”—Gastroenterology, 2006;130:1519-1526]. Mean of crying time was calculated on the first 7 consecutive days.

Of 21 infants included in the fMRI analysis, 11 were considered as having infant colic. Mean crying times: 122 min for all infants; 176 min for infants with colic; and 62 min for infants without colic.

### Results

The data shows a significant positive correlation between cerebral reactivity to cabbage-like odorant stimulation in the primary and secondary olfactory cortices (piriform cortex, hippocampus, orbito-frontal cortex (OFC) and cingulate cortex) and mean daily crying time at 5-6 weeks (FIGS. 2A, 2B and 2C). These data suggest that babies with colic may have a hypersensitivity to particular stimuli such as olfactory stimuli. These results are supported by a group comparison between babies with and without colic (FIG. 3). The hypersensitivity may be the result of, or may lead to, brain over-reactivity and subsequently overactivity in the areas of the brain involved in emotional/pain regulation, in particular, the amygdala.

### Example 2

### Effect of BL-999 Administration on Cerebral Activity

The following example is a randomized, double blind, placebo-controlled trial illustrative of B. longum ATCC BAA-999 reducing brain emotional reactivity.

Specific probiotic bacteria can improve gut symptoms of IBS, however, their efficacy in treating co-morbid anxiety or a depressive symptom in this population is unknown. B. longum ATCC BAA-999 was previously shown to normalize anxiety-like behavior and hippocampal neurotrophin levels in murine models of low-grade gut inflammation.

The aim of this experimental procedure was to evaluate the effects of B. longum ATCC BAA-999 on anxiety and depressive symptoms in patients with IBS and to study the underlying mechanisms. A randomized, double-blind, placebo-controlled, single center study was carried out in adult patients with IBS with diarrhea or mixed stool pattern (Rome III criteria) and mild to moderate anxiety and/or a depressive symptom.

There were no differences in demographics and baseline data between the two groups, except for HAD-D scores, which were higher in B. longum group (p=0.046). B. longum ATCC BAA-999 (1.0E+10 CFU daily) or placebo (maltodextrin) was administered daily for six weeks.

Validated questionnaires were used to assess anxiety and a depressive symptom (HAD score (Hospital Anxiety and Depression) and STAI (State-Trait Anxiety Inventory) score), IBS symptoms (adequate relief question, IBS Birmingham and Bristol scale), quality of life (SF-36) and somatization (PHQ-15) before administration, at the end of administration, and one month after the treatment (follow-up). This experimental design is shown in FIG. 4.

Brain activation patterns were assessed using the backward masked fear paradigm (fMRI), cognitive function (memory and concentration), serum BDNF and inflammatory markers, and gut microbiota profiles (16S rRNA Illumina). The fMRI paradigm utilized Blood Oxygenation Level Dependent (BOLD) activation in response to the presentation of emotional stimuli (fear and happy faces) that were masked by a neutral face, measured over four consecutive fMRI scan acquisitions in the scanner. The amygdala was selected as a priori region of interest. This analysis was performed on all subjects.

Results 44 patients were randomized, and 38 of them (B. longum ATCC BAA-999=18, placebo=20) completed the study. The results are shown in FIGS. 5-9. At six weeks, depression scores improved in patients treated with B. longum ATCC BAA-999 compared with placebo (RR 2.94, 95% Cl 1.05-8.23, p=0.01), and this beneficial effect was maintained at follow-up.

More patients treated with B. longum ATCC BAA-999 than placebo reported adequate relief of overall IBS symptoms (RR 2.1, 95% CI 1.15-3.83, p=0.02) but no statistically significant changes were found in the IBS Birmingham scores. The physical subdomain of quality of life improved in the group treated with B. longum ATCC BAA-999 compared with placebo (p=0.03, Mann-Whitney U=228.5), with trends for improvement in the mental subdomains of vitality and emotional role functioning.

The beneficial effect of B. longum ATCC BAA-999 on a depressive symptom was maintained at one month post-treatment, while IBS symptoms and quality of life returned to baseline.

Specifically, FIG. 6 shows that treatment with B. longum ATCC BAA-999 improved depression scores both by intention-to-treat analysis (ITT) and per protocol analysis (PP). The beneficial effect of the B. longum ATCC BAA-999 was maintained at one month post-treatment (follow-up visit, with both ITT and PP analysis).

FIG. 7 shows that, adjusting for baseline, depression improvement as a continuous variable was achieved in the B. longum ATCC BAA-999 group (ANCOVA, p=0.049). This beneficial effect was not maintained at one month post-treatment. Treatment with B. longum ATCC BAA-999 did not improve anxiety scores when analyzed as continuous variables.

FIGS. 8 and 9 show that there was a statistically significant improvement in SF-36 physical global domain, as well as in general physical health (Physical functioning) and problems with work or other daily activities (Role physical), in the B. longum ATCC BAA-999 group compared to placebo. Non-significant differences between treatment groups were observed in SF-36 mental global domain. However, when analyzing the mental subdomains, non-statistically significant trends for improvement in Vitality and Role emotional were observed in the B. longum ATCC BAA-999 treated group.

FIG. 2 shows that functional MRI revealed significant reductions from baseline in response to negative emotional stimuli in multiple brain areas involved in emotion processing, including amygdala, frontal and temporal brain regions (p<0.001), in patients treated with B. longum ATCC BAA-999 compared with placebo. Specifically, before treatment, there was no major difference in response to fear stimuli vs fixation between placebo and B. longum groups, except for greater engagement of the visual association and parietal cortices in B. longum group. However, at the end of the treatment, there was greater engagement of the amygdala, frontal, and temporal cortices and reduced engagement of occipital regions in placebo group.

No statistically significant differences were observed in anxiety, cognitive function, inflammatory markers, serum BDNF levels or gut microbiota profiles in patients treated with B. longum ATCC BAA-999 compared to placebo.

The results demonstrate that a six-week treatment with B. longum ATCC BAA-999 improves co-morbid depressive symptoms, overall gastrointestinal symptoms and quality of life in patients with IBS. This effect is associated with changes in the brain activation patterns in the amygdala and fronto-limbic regions, suggesting that reduction in limbic reactivity may underlie the beneficial effect of B. longum ATCC BAA-999.

The results from Example 2 therefore demonstrate that administration of B. longum ATCC BAA-999 can reduce brain activity in areas involved in emotional/pain regulation, particularly the amygdala. According to Example 1, babies with colic have a pronounced over-reactivity in the amygdala, compared with babies that do not have colic. The data shows that administration of B. longum ATCC BAA-99 can be used to reduce cerebral activity in the amygdala, and hence can provide an effective treatment for colic.

1. A method for the treatment or prophylaxis of infant colic comprising administering a composition comprising Bifidobacterium longum ATCC BAA-999 to an infant in need of same.

2. A method for reducing cerebral activity in an infant with colic comprising administering a composition comprising Bifidobacterium longum ATCC BAA-999 to an infant in need of same.

3. (canceled)

4. A method of reducing frequency and/or duration of episodes of irritability, fussing and/or crying in a subject with infant colic, comprising administering Bifidobacterium longum ATCC BAA-999 or a composition thereof to the infant.

5. Method according to claim 1, wherein the infant has been identified as having an increased cerebral activity.

6. Method according to claim 1, wherein the method comprises administration of Bifidobacterium longum ATCC BAA-999 to the infant in an amount of: about 1×10⁸ CFU or more per day.

7. Method according to claim 1, wherein the method comprises administration of Bifidobacterium longum ATCC BAA-999 to the infant in an amount of: about 1×10⁸ to about 5×10¹¹ CFU per day.

8-12. (canceled)

13. Method according to claim 1, wherein the composition is in a form selected from the group consisting of an infant formula, an infant supplement and a maternal lactation supplement.

14. Method according to claim 1, wherein the composition does not comprise a Lactobacillus probiotic.

15-16. (canceled)

17. Method according to claim 2, wherein the infant has been identified as having an increased cerebral activity.

18. Method according to claim 2, wherein the method comprises administration of Bifidobacterium longum ATCC BAA-999 to the infant in an amount of: about 1×10⁸ CFU or more per day.

19. Method according to claim 2, wherein the method comprises administration of Bifidobacterium longum ATCC BAA-999 to the infant in an amount of: about 1×10⁸ to about 5×10¹¹ CFU per day.

20. Method according to claim 4, wherein the infant has been identified as having an increased cerebral activity.

21. Method according to claim 4, wherein the method comprises administration of Bifidobacterium longum ATCC BAA-999 to the infant in an amount of: about 1×10⁸ CFU or more per day.

22. Method according to claim 4, wherein the method comprises administration of Bifidobacterium longum ATCC BAA-999 to the infant in an amount of: about 1×10⁸ to about 5×10¹¹ CFU per day.

23. Method according to claim 2, wherein the composition is in a form selected from the group consisting of an infant formula, an infant supplement and a maternal lactation supplement.

24. Method according to claim 2, wherein the composition does not comprise a Lactobacillus probiotic.

25. Method according to claim 4, wherein the composition is in a form selected from the group consisting of an infant formula, an infant supplement and a maternal lactation supplement.

26. Method according to claim 4, wherein the composition does not comprise a Lactobacillus probiotic.
